(19) United States
(12) Patent Application Publication
(10) Pub. No.: US 2022/0020499 A1
(43) Pub. Date: Jan. 20, 2022

(54) METHOD AND SYSTEM FOR DETERMINING A RELATIVE RISK FOR LACK OF GLYCEMIC CONTROL FOR A PLURALITY OF PATIENTS

(71) Applicant: mySugr GmbH, Wien (AT)

(72) Inventors: Lukas Schuster, Wien (AT); Jan Wrede, Salzgitter-Bad (DE); Richard P. Biven, Wien (AT); Michèle Theissen, Wien (AT)

(21) Appl. No.: 17/487,940

(22) Filed: Sep. 28, 2021

Related U.S. Application Data
(63) Continuation of application No. PCT/EP2020/058697, filed on Mar. 27, 2020.

(30) Foreign Application Priority Data
Mar. 29, 2019 (EP) 19 166 408.5

(51) Int. Cl. G16H 50/30; A61B 5/145

(52) U.S. Cl. CPC G16H 50/30; A61B 5/14532

(57) ABSTRACT

A method for determining relative risk for lack of glycemic control for at least two patients. First and second sets of glucose readings are provided and assigned to first and second patients, respectively. The first and second sets of glucose readings are collected by conducting first and second different frequencies of measurements, respectively. Evaluation parameters and evaluation values are determined. First and second risk scores are determined and assigned to the first and second patients, respectively. A relative risk for lack of glycemic control is determined by comparing a first total risk score and a second total risk score. The relative risk indicates that the risk for lack of glycemic control is higher for one of the two patients.

### RELATED APPLICATIONS

This application is a continuation of PCT/EP2020/058697, filed Mar. 27, 2020, which claims priority to EP 19 166 408.5, filed Mar. 29, 2019, the entire disclosures of both of which are hereby incorporated herein by reference.

### BACKGROUND

The present disclosure refers to a computer-implemented method and a system for determining a relative risk for lack of glycemic control for a plurality of patients. A computer program product is also referred to.

Glucose measurement data collected for one or more patients may be analyzed for providing caregivers information as to whether some patient needs assistance or support. There are different types of measurements conducted for gathering glucose measurement data, such as blood glucose measurement data. Also, continuous glucose monitoring (CGM) may be applied. For such type of measurement the frequency of measurement events (1/T) is very high, with the typical measurement time interval T approximately ranging from 5 to 15 minutes, which allows analyses with high resolution. In contrast, for patients performing so-called spot monitoring (blood) glucose measurements (SPM), the number of samples per day is comparably low, e.g., around 3 to 8 samples, and mostly unevenly distributed in time. Spot monitoring blood glucose (SPM) measurement may also be referred to as self-monitored blood glucose (SMBG) measurement.

U.S. Publication No. 2009/0171589 A1 describes computing an average daily risk range (ADRR). Parameters low blood glucose index (LBGI) and high blood glucose index (HBGI) are referred to in this document with indication of further references.

U.S. Publication No. 2015/0095042 A1 discloses classifying a patient as having a first, second or third risk of being hypoglycemic in the future based on a value (e.g., LBGI value) which is calculated based on blood glucose levels and displaying the classification on a display. Also, it may be determined whether a pattern is present in a patient's blood glucose data. The order of display of recognized patterns may be prioritized.

EP 5 062 615 B1 refers to a system for monitoring, diagnosing and treating medical conditions of a plurality of remotely located patients comprising a central data processing system, means for obtaining patient data from remotely located patient monitoring systems, means for analyzing the obtained patient data from each respective patient monitoring system to identify medical conditions of each respective patient; means for displaying identified patient medical conditions for each respective patient in selectable prioritized order according to medical severity. Communication between portable patient monitors (PPMs) with a central data processing system referred to as a Physicians Access Center server (“PAC server”) is described.

WO 2016/182972 A1 discloses categorization of a plurality of patients into diabetes risk categories for caregivers.

U.S. Publication No. 2006/0253303 A1 relates to providing a multiple patient monitoring system. An overview chart is provided, wherein each data point represents one corresponding patient and indicates the control value, e.g., a mean value, and the elapsed time period since the collection date of the set of measurements most recently collected for the patient. Furthermore, each icon may indicate compliance of the corresponding patient with the prescribed measurement regimen. The clinician can thus view and manage the medical priorities of an entire group of patients simultaneously. It also allows the clinician to communicate proactively with unmotivated patients who have lost contact with the clinician before these patients develop urgent medical needs.

WO 2017/030961 A1 discloses a patient prioritization module which sorts and prioritizes the healthcare provider's patients based on the alert status of each of the patient. The alert sort algorithm pre-assigns a weight factor for each glucose condition and for each possible state of a given glucose condition.

WO 2007/081853 A2 pertains to a system, a computer program product, a method and an algorithm for the evaluation of blood glucose. The method employs routine self-monitoring blood glucose data collected over a period of 2-6 weeks, based on a theory of risk analysis of blood glucose data. An Average Daily Risk Range (ADRR) is computed as a measure of overall glucose variability. Further, the glucose variability in the hypoglycemic range via a Low BG Index (LBGI) and the glucose variability in the high BG range via High BG Index (HBGI) followed by a combination of the two indices into a single variability display may be estimated separately.

The document Kovatchev et al., Risk analysis of blood glucose data: a quantitative approach to optimizing the control of insulin dependent diabetes, J. Theor. Med. 3, 1-10 (2000), pertains to quantitative tools for online assessment of the quality of an optimization based on self-monitoring of blood glucose.

The document Kovatchev, Metrics for glycemic control from HbA1c to continuous glucose monitoring, Nature Rev. Endocrinology 13, 425-436 (2017) refers to the assessment, quantification and optimal control of glucose fluctuations in diabetes mellitus, focusing on markers of average glycaemia and the utility and shortcomings of HbA1c as a gold-standard metric of glycemic control.

The document Kovatchev et al., Evaluation of a New Measure of Blood Glucose Variability in Diabetes, Diabetes Care 29:11, 2433-2438 (2006) pertains to average daily risk range (ADRR) as a variability measure computed from routine self-monitored blood glucose (SMBG) data.

### SUMMARY

The present disclosure provides a computer-implemented method and a system for determining a relative risk for lack of glycemic control for a plurality of patients, wherein the method and the system can be applied for reliable determination of the relative risk for lack of glycemic control for glucose measurement data collected by conducting different measurement conditions.

According to an aspect, a computer-implemented method for determining a relative risk for lack of glycemic control for at least two patients is provided, the method, in a data processing device having one or more data processors and a data storage device connected to the one or more data processors, comprising: providing a first set of glucose measurement data assigned to at least one a first patient, the first set of glucose measurement data collected by conducting a first frequency of measurement events; providing a second set of glucose measurement data assigned to a second patient, the second set of glucose measurement data collected by conducting a second frequency of measurement events which is different from the first frequency of measurement events; providing a plurality of evaluation parameters in the data storage device, each of the evaluation parameters assigned to at least one of the first frequency of measurement events and the second frequency of measurement events; determining, depending on the first frequency of measurement events, at least one first evaluation parameter from the plurality of evaluation parameters in the data storage device; determining, depending on the second frequency of measurement events, at least one second evaluation parameter which is different from the at least one first evaluation parameter from the plurality of evaluation parameters in the data storage device; determining, from the first set of glucose measurement data, a first evaluation parameter value assigned to the first patient for the at least one first evaluation parameter; determining, from the second set of glucose measurement data, a second evaluation parameter value assigned to the second patient for the at least one second evaluation parameter; determining a first risk score assigned to the first patient and a second risk score assigned to the second patient from the first evaluation parameter value and second evaluation parameter value, respectively; and determining a relative risk for lack of glycemic control by comparing a first total risk score comprising the first risk score and a second total risk score comprising the second risk score, the relative risk indicating the risk for lack of glycemic control being higher for one of the first patient and the at least one second patient.

According to another aspect, a system for determining a relative risk for lack of glycemic control for at least two patients is provided, the system comprising a data processing device having one or more data processors and a data storage device connected to the one or more data processors, wherein the data processing device is configured to: provide a first set of glucose measurement data assigned to a first patient, the first set of glucose measurement data collected conducting a first frequency of measurement events; provide a second set of glucose measurement data assigned to at least one a second patient, the second set of glucose measurement data collected conducting a second frequency of measurement events which is different from the first frequency of measurement events; provide a plurality of evaluation parameters in the data storage device, each of the evaluation parameters assigned to at least one of the first frequency of measurement events and the second frequency of measurement events; determine, depending on the first frequency of measurement events, at least one first evaluation parameter from the plurality of evaluation parameters in the data storage device; determine, depending on the second frequency of measurement events, at least one second evaluation parameter which is different from the at least one first evaluation parameter from the plurality of evaluation parameters in the data storage device; determine, from the first set of glucose measurement data, a first evaluation parameter value assigned to the first patient for the at least one first evaluation parameter; determine, from the second set of glucose measurement data, a second evaluation parameter value assigned to the second patient for the at least one second evaluation parameter; determine a first risk score assigned to the first patient and a second risk score assigned the second patient from the first evaluation parameter value and second evaluation parameter value, respectively; and determine a relative risk for lack of glycemic control by comparing a first total risk score comprising the first risk score and a second total risk score comprising the second risk score, the relative risk indicating the risk for lack of glycemic control being higher for one of the first patient and the second patient.

Further, a computer program product is provided, comprising program code configured to, when loaded into a computer having one or more processors, perform the implemented method for determining a relative risk for lack of glycemic control for at least two patients.

The relative risk indicating the risk for lack of glycemic control may give indication that a patient or a group of patients is in higher or more urgent need for assistance or coaching than some other patient or group of patients due to different risk score. Such information can be provided to some health care provider or professional, for example, by outputting audio and/or video data indicating the relative risk for lack of glycemic control.

The first set of glucose measurement data and the second set of glucose measurement data may be received from at least one of spot monitoring blood glucose (SPM) measurement which also be referred to as self-monitored blood glucose (SMBG) measurement and continuous glucose monitoring (CGM). The data processing device may, e.g., receive the first set of glucose measurement data and the second set of glucose measurement data wirelessly from handheld portable glucose meters (for making spot monitoring blood glucose measurements) and/or from continuous glucose monitoring devices (for continuous glucose monitoring).

For determining the first evaluation parameter value all measurement data or a subset of measurement data from the first set of glucose measurement data provided may be analyzed. The same applies to the determining of the second evaluation parameter value, analysis of all measurement data or a subset of measurement data from the second set of glucose measurement data provided may be conducted. For example, no data older than 30 days or older than 14 days may be analyzed for determining the relative risk for lack of glycemic control.

In one embodiment, the first set of measurement data may comprise or consist of data not older than 30 days or not older than 14 days, and the second set of measurement data may comprise or consist of data not older than 30 days or not older than 14 days for determining the relative risk for lack of glycemic control.

Different frequencies of measurement events are assigned different sets of evaluation parameters in the data storage device. Such assignment may be provided by assignment data stored in the data storage device. The assignment data may be stored in the data storage device in response to a user input indicating the assignment. Alternatively, the assignment data may be received from some external source such as a remote server device or a remote input device. After receiving the assignment data in a processor of the data processing device, the evaluation parameter(s) assigned to the first and the second frequency of measurement events may be determined from the assignment data. The different frequencies of measurement events may be assigned a single evaluation parameter or a group of evaluation parameters.

The method may comprise at least one of the following: providing a first set of glucose measurement data collected in a first plurality of spot monitoring glucose measurement events applying the first frequency of measurement events; and providing a second set of glucose measurement data collected in a second plurality of spot monitoring glucose measurement events applying the second frequency of measurement events.

Depending on the frequency of measurement events conducted in the glucose measurement different evaluation parameters are applied for determining the relative risk for lack of glycemic control. Still, the risk score determined for the different sets of glucose measurement data each assigned to one or more patients can be compared for determining the relative risk for lack of glycemic control. Based on such information about the relative risk some care keeper can decide whether some patient is more in need for consultation or support then some other patient.

For at least one of the first and second set of glucose measurement data the following may be provided: determining one or more consecutive glucose measurement values being collected subsequently to collecting a preceding glucose measurement value in a preceding measurement event, the one or more measurement values collected in one or more consecutive measurement events within a predetermined time window subsequently to the preceding measurement event; and excluding the one or more consecutive glucose measurement values from the determining of the first/second evaluation parameter value. For example, only a very first glucose measurement value (collected in the preceding measurement event) may be taken into account for determining of the first/second evaluation parameter value, the one or more consecutive glucose measurement values collected subsequently are omitted. Alternatively, a mean or median value may be determined for the one or more consecutive glucose measurement values and the preceding glucose measurement value. Following, the mean or the median value may be taken into account for determining of the first/second evaluation parameter value. In an embodiment, the predetermined time window may be about 30 min or 60 min.

In the data storage device time window data may be stored, for example, in response to a user input indicating a time window. In the process of determining the one or more consecutive glucose measurement values, the time window data may be retrieved by one of the processors from the data storage device. Following, the time window defined by the time window data is determined by the processor and applied. In addition or as an alternative, number data may be stored in the data storage device, the number data being indicative of a predetermined number of consecutive glucose measurement values to be excluded. The number data may be received in the one or more processors from the data storage device and applied in the process of excluding the one or more consecutive glucose measurement values.

The method may further comprise the following: determining, from the first set of glucose measurement data, a plurality of first evaluation parameter values assigned to the first patient for a plurality of first evaluation parameters; and determining, from the second set of glucose measurement data, a plurality of second evaluation parameter values assigned to the second patient for a plurality of second evaluation parameters. The first total risk score and the second total risk score may be determined by summing up first and second risk scores for the plurality of first evaluation parameter values and the plurality of second evaluation parameter values, respectively. The total risk scores are compared for determining the relative risk for lack of glycemic control.

At least one common evaluation parameter may be provided in both the plurality of first and the plurality of second evaluation parameters.

The method may provide the following: the first set of glucose measurement data is assigned to a first patient having a first type of diabetes; and the second set of glucose measurement data is assigned to a second patient having a second type of diabetes. Further, the method may comprise selecting at least one of the at least one first evaluation parameter and the at least one second evaluation parameter depending on the first type of diabetes and the second type of diabetes, respectively. In the data storage device additional assignment data may be stored, for example, in response to a user input indicating assignment of the first set of glucose measurement data to the first patient having the first type of diabetes, and assignment of the second set of glucose measurement data to the second patient having the second type of diabetes. In addition or as an alternative, the additional assignment data may be indicative of the assignment between the different types of diabetes on one side and different sets of evaluation parameters on the other side. In the process of determining the risk score(s), the additional assignment data may be retrieved from the data storage device. Following, the assignment of evaluation parameter(s) defined by the additional assignment data is determined from the additional assignment data by a processor of the data processing device and applied.

The second patient may be different from the first patient. Alternatively the first and second set of glucose measurement data may be collected for one and the same patient, but will refer to different measurement time windows for such patient. In such alternative example the first and second risk scores refer to an analysis of glucose measurement data for one and same person (patient), but the first and second measurement data collected at different measurement time windows for such patient who is referred to as first and second patient for differentiating the first and second measurement data collected at different times.

The first evaluation parameter and the second evaluation parameter may be selected from the following group of evaluation parameters: low blood glucose index, high blood glucose index, warning signs, mean blood glucose, median blood glucose, stability index, and total daily blood glucose variation determined by summing up peak risks of hypoglycemia and hyperglycemia events per day. The stability index is the mean value of the glucose measurement data for the patient divided by standard deviation.

The first evaluation parameter or the plurality of first evaluation parameters may be selected from the following group of evaluation parameters: low blood glucose index; high blood glucose index, warning signs, mean blood glucose, and median blood glucose.

The second evaluation parameter or the plurality of second evaluation parameters may be selected from the following group of evaluation parameters: low blood glucose index; high blood glucose index, stability index, and total daily blood glucose variation determined by summing up peak risks of hypoglycemia and hyperglycemia events per day.

In one embodiment, the plurality of first evaluation parameters may comprise low blood glucose index (LBGI); high blood glucose index (HBGI), warning signs (WS), and median blood glucose (median). The plurality of second evaluation parameters may comprise low blood glucose index (LBGI); high blood glucose index (HBGI), stability index (SI), and total daily blood glucose variation determined by summing up peak risks of hypoglycemia and hyperglycemia events per day (ADRR). The risk scores for the plurality of first evaluation parameter values and the plurality of second evaluation parameter values, respectively, may be determined according to Table 1.

TABLE 1

| | 0 (NO | 1 (LOW | 2 (MEDIUM | 3 (HIGH |
|---|---|---|---|---|
| Risk scores | RISK) | RISK) | RISK) | RISK) |
| LBGI | ≤1 | >1 & ≤2.5 | >2.5 & ≤5 | >5 |
| HBGI | ≤1 | >1 & ≤4.5 | >4.5 & ≤9 | >9 |
| SI | ≥3 | <3 & ≥2 | <2 & ≥1 | <1 |
| ADRR | ≤19 | >19 & ≤30 | >30 & ≤40 | >40 |
| Warning signs | ≤15% | >15% & ≤40% | >40% & ≤65% | >65% |
| Median | ≥70 & ≤140 | >140 & ≤154 | >154 & ≤169 | >169 |
| | | | or <70 | |

The parameter value for warning signs may be calculated as warning sign risk variable (WSRV) according to equation (1):

$$W S R V = \frac{{\sum }_{i} ( {w}_{i} \cdot {x}_{i} )}{{\sum }_{i} {w}_{i}} * 100 , \qquad ( 1 )$$

wherein index i denotes events, weight for the event with index i is denoted with wᵢ, and variable xᵢ represents a Boolean value whether (Boolean value=1) or not (Boolean value=0) the event has occurred.

The events may, e.g., comprise one or more of the following: Repetitive Hypoglycemia <70 mg/dl; Repetitive Hyperglycemia >300 mg/dl; Repetitive severe Hypoglycemia <50 mg/dl; Severe Hypoglycemia <40 mg/dl; and Severe Hyperglycemia >400 mg/dl; wherein Repetitive Hypo <70 mg/dl (Rep Ho<70) refers to two SMBG readings below 70 mg/dl in two consecutive days in the first set of glucose measurement data; Repetitive Hyperglycemia >300 mg/dl (Rep He >300) refers to more than three SMBG readings above 300 mg/dl in the first set of glucose measurement data; Repetitive severe Hypoglycemia <50 mg/dl (Rep SHo<50) refers to two SMBG readings below 50 mg/dl in two days in the first set of glucose measurement data; Severe Hypoglycemia <40 mg/dl (SHo<40) refers to one SMBG reading below 40 mg/dl in the first set of glucose measurement data; and Severe Hyperglycemia >400 mg/dl (SHe>400) refers to one SMBG reading above 400 mg/dl in the first set of glucose measurement data.

Weights wᵢ may be assigned to the events depending on the type of diabetes of the first patient according to Table 2.

TABLE 2

| | | TYPE 2/ |
|---|---|---|
| | TYPE 1/ | i MODY/ |
| Event | LADA | OTHERS |
| Rep Ho <70 | 4 | 4 |
| Rep He >300 | 5 | 5 |
| Rep SHo <50 | 5 | 5 |
| SHo <40 | 2 | 2 |
| SHe >400 | 3 | 1 |

For example, if the type of diabetes of the first patient is type 1 diabetes (TYPE 1) or latent autoimmune diabetes in adults (LADA) a weight of 3 may thus be assigned to the event Severe Hyperglycemia >400 mg/dl (SHe>400) while, if the type of diabetes of the first patient is type 2 diabetes (TYPE 2) or Maturity Onset Diabetes of the Young (MODY) or OTHERS a weight of 1 may be assigned to the event Severe Hyperglycemia >400 mg/dl (SHe>400).

In one embodiment the first patient has type 1 or type 2 diabetes and the second patient has type 1 or type 2 diabetes. In one embodiment, one of the first and second patient has type 1 diabetes and the other one has type 2 diabetes.

At least one of the first total risk score and the second total risk score, being above a predefined threshold value, may be indicative of at least one of a need for therapy review, and therapy adjustment for the first patient and the second patient, respectively. As an alternative or in addition, at least one of the first total risk score and the second total risk score being above a predefined threshold value may trigger an electronic message being sent to the first patient and the second patient, respectively.

The predefined threshold value may be stored in and retrieved from the data storage device for determining whether or not there is a need of therapy review and/or therapy adjustment and/or for triggering an automatic electronic message being sent to the patient(s) whose score(s) exceed(s) the threshold value.

In a further embodiment, the following may be provided:
a) a first maximum achievable total risk score and a first threshold value are predefined, and a ratio of the first total risk score and the first maximum achievable total risk score being above the first predefined threshold
   is indicative of at least one of need for therapy review and therapy adjustment for the first patient, and/or
   triggers an electronic message being sent to the first patient
   wherein the first predefined threshold value is above 0.5, preferably above 0.7, more preferably above 0.8 or 0.9; and/or
b) a second maximum achievable total risk score and a second threshold value are predefined and a ratio of the second total risk score and the second maximum achievable total risk score being above the second predefined threshold
   is indicative of at least one of need for therapy review and therapy adjustment for the second patient, and/or
   triggers an electronic message being sent to the second patient
   wherein the second predefined threshold value is above 0.5, preferably above 0.7, more preferably above 0.8 or 0.9.

The ratio of the first total risk score and the first maximum achievable total risk score being above the first predefined threshold value is indicative of at least one of need for therapy review and therapy adjustment for the first patient. In addition or as an alternative, the ratio of the first total risk score and the first maximum achievable total risk score being above the first predefined threshold value may trigger an electronic message being sent to the first patient.

Similarly, the ratio of the second total risk score and the second maximum achievable total risk score being above the second predefined threshold value is indicative of at least one of need for therapy review and therapy adjustment for the second patient. In addition or as an alternative, the ratio of the second total risk score and the second maximum achievable total risk score being above the second predefined threshold value may trigger an electronic message being sent to the second patient.

The predefined first maximum achievable total risk score and the predefined first threshold value may be stored in and retrieved from the data storage device for determining whether or not there is a need of therapy review and/or therapy adjustment for the first patient and/or for triggering an automatic electronic message being sent to the first patient if the first threshold value is exceeded. The predefined second maximum achievable total risk score and the predefined second threshold value may be stored in and retrieved from the data storage device for determining whether or not there is a need of therapy review and/or therapy adjustment for the second patient and/or for triggering an automatic electronic message being sent to the second patient if the second threshold value is exceeded.

The message may comprise one or more of i) teaching material on how to improve glycemic control, ii) a meeting request with a health care professional, iii) a prompt to increase the measurement frequency. In one embodiment a prompt to increase the measurement frequency is sent only if the frequency of measurement events of the patient is lower than a predefined frequency threshold stored in the data storage device.

Output data indicating information about the relative risk may be output through a display device connected to the data processing device.

The embodiments disclosed for the method above may apply to the system mutatis mutandis.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 is a schematic representation of a system for determining a relative risk for lack of glycemic control for a plurality of patients;

FIG. 2 is a schematic representation for a method for determining a relative risk for lack of glycemic control for a plurality of patients;

FIG. 3 is a table comprising weights for weighting warning signs for different diabetic types;

FIG. 4 is a table summarizing different groups of diabetic patients and corresponding metrics;

FIG. 5 is a table summarizing the scoring system and comprising different risk metrics;

FIG. 6 is a table comprising the risk scores for 23 exemplary patients; and

FIGS. 7a to 7d are four graphs of blood glucose (BG) values depending on time of recording for patients of different groups.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

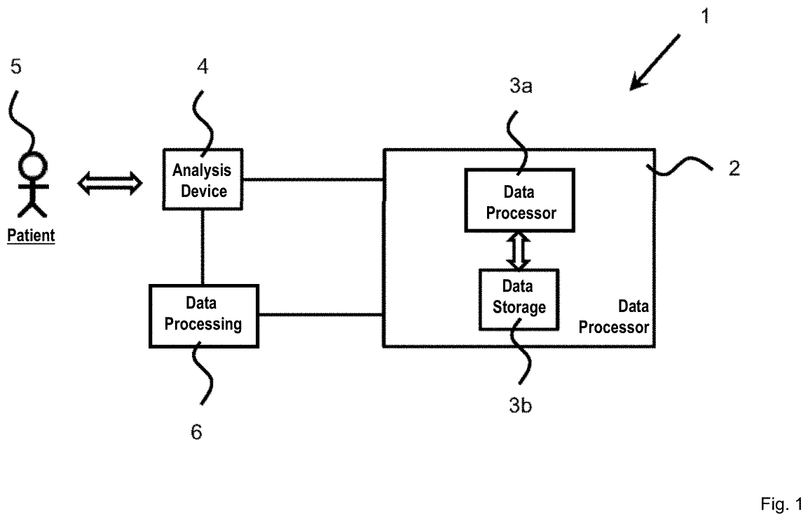

FIG. 1 shows a schematic representation of a system 1 for determining a relative risk for lack of glycemic control for a plurality of patients, the system 1 comprising a data processing device 2 having one or more data processors 3a. The data processing device 2 which may be implemented on a mobile or non-mobile device further comprises a data storage device 3b functionally connected to the one or more data processors 3a for exchanging electronic data. From a measurement or analysis device 4, the data processing device (or “data processor”) 2 receives measurement data indicative of a glucose level for one or more patients 5. In a typical use case, measurement data indicative of a glucose level for at least two patients will be received. The measurement data for different patients may be received from different measurement or analysis devices used by the different patients. For example, each patient may be using their personal measurement or analysis device for collecting the measurement data indicative of the patient's (blood) glucose level. Alternatively, the measurement data, which may also be referred to as readings, may be received from one or more other data processing devices 6 which received the measurement data before, for example, from a plurality of measurement or analysis devices.

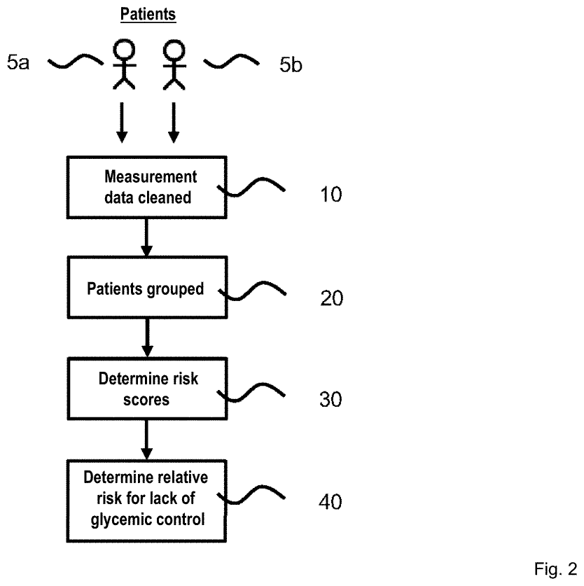

FIG. 2 shows a schematic representation for a computer-implemented method for determining a relative risk for lack of glycemic control for at least two patients 5a, 5b. The determining of the relative risk for lack of glycemic control comprises assigning risk metrics to the patients 5a, 5b. In a (optional) first step 10, measurement data collected in SMBG (self-monitoring of blood glucose) which may also be referred to as spot-monitoring (SPM) measurement are cleaned. The measurement data representing SMBG readings or measurement (events) may have to be cleaned, since some of the patients 5 may have recorded several SMBG readings within a short time period. This is sometimes referred to as “binge monitoring,” which may be due to intensified control in time periods of glycemic excursion or activities that pose an increased risk, such as sports. SMBG readings for one and the same patient that take place within a predetermined time period of, for example, 30 minutes or less are removed from the input data by the cleaning. Alternatively, a predetermined time period of 60 minutes may apply. Thus, the data are not skewed around such time periods.

Following, in step 20 the at least two patients 5a, 5b are grouped. For example, each patient may be assigned to one of the following groups: G₁—patients that record less than two SMBG readings per day on average, and G₂—users that record two or more SMBG readings per day on average. In an embodiment, the groups G₁ and G₂ may represent SMBG readings of less than 2 (or less than 5) and ≥2 (or ≥5), respectively, per day in 14 days out of 30 consecutive days in which most recordings or measurements were conducted. The SMBG readings of groups G₁ and G₂ represent a first frequency of measurement events and a second frequency of measurement events, respectively. In this example, the second frequency of measurement events is higher than the first frequency of measurement events.

As an example, if a patient checks their SMBG a minimum of two times per day for at least 14 days within the last 30 days, they will be assigned to the group G₂. Note that such measure allows for days where the patient records less frequent or not at all as long as they have at least fourteen days meeting the above requirement. Also, if another patient records at least 4 SMBG data every other day for 30 days, they will also be assigned to the group G₂.

Alternatively, the groups G₁ and G₂ can also represent less than 2 (or less than 5) and ≥2 (or ≥5) SMBG readings, respectively, per day in 7 days out of 14 consecutive days in which most recordings were performed. The two-week time span may be beneficial because it looks at only the most recent measurement data. Measurement events that take place 30 days prior might not be related to the patient's current therapy regiment or easy to remember. However, on the other hand, 7 out of 14 days is less data for analysis.

Referring to FIG. 2, in step 30 for the patients 5 in groups G₁ and G₂ risk scores are determined. Following, in step 40 a relative risk for lack of glycemic control is determined by comparing the (total) risk scores for the patients 5a, 5b assigned to groups G1 and G2.

Since the groups G₁ and G₂ are representing patients having applied different frequencies for SMBG readings or measurements, different metric(s) or a different plurality of metrics (which may have at least one metric in common) is applied for the patients in the two different groups for determining individual risk scores. The application of different metrics allows for comparing the risk score determined for the patients in groups Gland G₂, thereby determining a relative risk for lack of glycemic control. Such relative risk may be higher for a patient of one or the other group depending on the actual SBMG readings or measurements. Also, more than two patients may be compared. In case of a number of i (i >2) patients, the patients may be assigned to groups Gⱼ (j≤i).

A first metric which may referred to as ADRR (Average Daily Risk Range) is proposed, ADRR constituting an adequate risk measure capable of comparing risks for lack of glycemic control for patients 5 falling in the different groups G₁ and G₂.

In order to calculate the risk for high and low SMBG values, HBGI (High Blood Glucose Index) and LBGI (Low Blood Glucose Index) transformation equations (2) and (3), as well as published risked values are employed (cf. Kovatchev et al. (2000)).

$$\begin{matrix}L B G I = \frac{1}{n} {\sum }_{i = 1}^{n} rl ( {x}_{1} ) & ( 2 ) \\ H B G I = \frac{1}{n} {\sum }_{i = 1}^{n} r h ( {x}_{1} ) & ( 3 ) \\ where & \\ rl ( BG ) = \{ \begin{matrix}r ( BG ) & if f ( BG ) < 0 \\ 0 & otherwise\end{matrix} & ( 4 ) \\ rh ( BG ) = \{ \begin{matrix}r ( BG ) & if f ( BG ) > 0 \\ 0 & otherwise\end{matrix} & \end{matrix}$$

with BG denoting the recorded blood glucose value in a SMBG recording,

r(BG)=10·ƒ(BG)² (5)

and

ƒ(BG)=1.509·(ln(BG)^{1.084}−5.381) (6)

if the values for BG are measured in units of milligrams per deciliter, or

ƒ(BG)=1.794·(ln(BG)^{1.026}−1.861) (6′)

if the values for BG are measured in units of millimoles per liter. The function r can be interpreted as a measure of the risk associated with a certain BG level. The function rl de-pending on BG represents a low risk and the function rh represents a high risk. The function ƒ is a continuous function defined on a BG range of 1.1 to 33.3 millimoles per liter. Its form can be obtained as described in Kovatchev et al. (2000).

LBGI and HBGI are known to provide an assessment of patients' glycemic control covering both the risk for hypoglycemia and the risk of hyperglycemia, respectively. LBGI and HBGI are not time-dependent variables. They are a transformation and normalization of SMBG values to provide an equal risk scale for hypoglycemia and hyperglycemia. They are further known to gradually increase with the extent and frequency of hypoglycemic and hyperglycemic events. Therefore, LBGI and HBGI were applied for all patients 5 in each group G₁ and G₂, allowing for comparable metrics for both low and high frequency SMBG patients 5.

Based on the LBGI and HBGI values, the metric ADRR is calculated. ADRR provides a risk assessment of the total daily blood glucose variation, i.e., the sum of peak risks of hypoglycemia and hyperglycemia events per day. The ADRR is calculated as follows:

$$\begin{matrix}ADRR = \frac{1}{M} {\sum }_{i = 1}^{M} ( L {R}^{i} + H {R}^{i} ) \\ where & ( 7 ) \\ L {R}^{i} = max ( rl ( {x}_{1}^{i} ) , rl ( {x}_{2}^{i} ) , … , rl ( {x}_{{n}_{i}}^{i} ) ) , & ( 8 ) \\ H {R}^{i} = max ( r h ( {x}_{1}^{i} ) , rh ( {x}_{2}^{i} ) , … , rh ( {x}_{{n}_{i}}^{i} ) ) . & \end{matrix}$$

The number of readings for day i is denoted with nᵢ and the blood glucose data points for day i with x₁ⁱ, x₁ⁱ, . . . , xₙ_{ᵢ}ⁱ (cf. WO 2007/081853 and Kovatchev et al. (2006)). M is the total number of days for which the ADRR is calculated.

For calculating the ADRR, a number of two readings per day can be sufficient. Alternatively, a minimum of three (or five) SMBG readings is used to calculate the ADRR.

The stability index (SI) is the mean value of the patient's glucose measurement data divided by the standard deviation (SI=μ/σ). A minimum of two SBGM readings or measurements a day is required to calculate a reasonable standard deviation. Alternatively, a minimum of three (or five) SMBG readings is used to calculate the SI.

Since both ADRR and SI require a minimum reading count for usage, two further metrics representing comparable risk for low frequency patients (group G₁) have been devised. These metrics will be discussed next. As HBGI and LBGI do not require a minimum number of SMBG readings, HBGI and LBGI are used for all patients 5 (G₁ and G₂) in order to find the risk of high and low glycemic excursion.

As an equivalent metric for ADRR, the median of the SMBG values of the patients 5 of the group G₁ can be employed. In one embodiment, the mean of the SMBG values of the patients 5 of the group G₁ can be employed. However, the median is less sensitive to outliers than the mean.

As an equivalent for SI, warning signs (WS) are applied as a way to create a G₁ risk score that is comparable to the SI risk score of the group G₂. If a patient meets the requirements of a certain percentage of WS, they are considered as lacking stability. The WS can be weighted with healthcare providers (based on the diabetic type) to determine the appropriate risk as follows.

The parameter value for warning signs may be calculated as warning sign risk variable (WSRV) which is calculated according to equation (1) above.

In the following, an example for the calculation of the WSRV for a patient is provided. The patient for the last month obtained readings meeting the criteria of Rep Hypo (BG<70 mg/dl) and Rep Severe Hypo (BG<50 mg/dl).

The patient neither experienced any hyperglycemic event nor any severe hypoglycemic event. The following values were obtained:

| Event (also called item) | Weight wᵢ | xᵢ |
|---|---|---|
| Rep Hypo <70 | 4 | 1 |
| Rep Hyper >300 | 5 | 0 |
| Rep SHypo <50 | 5 | 1 |
| Severe Hypo <40 | 2 | 0 |
| Severe Hyper >400 | 3 | 0 |

Plugging the values into equation (1) yields:

$$W S R V = \frac{4 \cdot 1 + 5 \cdot 0 + 5 \cdot 1 + 2 \cdot 0 + 3 \cdot 0}{4 + 5 + 5 + 2 + 3} * 100 = \frac{9}{19} * 100 = 47.4 \% . \qquad ( 9 )$$

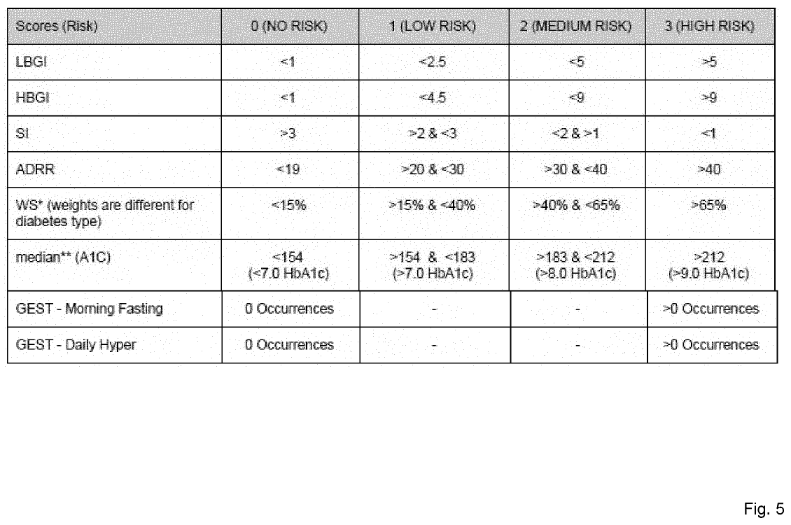

Following the table in FIG. 5, the patient falls into the Medium Risk category regarding the warning signs. Thus, with respect to WS Risk or SMBG Magnitude Metric (MM), the patient is assigned a score of 2. The patient also received a Medium Risk score for Low SMBG Metric (LM)=2, No Risk for High SMBG Metric (HM)=0, and a Low Risk=1 for Average Metric (AM) (e.g., ADRR or median).

Therefore, the patient's Overall Risk Score is:

$$Overall Risk Score = \frac{L M + H M + M M + A M}{( max ( L M ) + max ( H M ) + max ( M M ) + max ( A M )} = \frac{2 + 0 + 2 + 1}{3 + 3 + 3 + 3} = 0.4 \; 17 \qquad ( 10 )$$

A patient's Overall Risk Score can also be described as the ratio of the patient's total risk score and the maximum achievable total risk score for such patient.

The weights are set up in a way that they can be changed to meet the patient's needs.

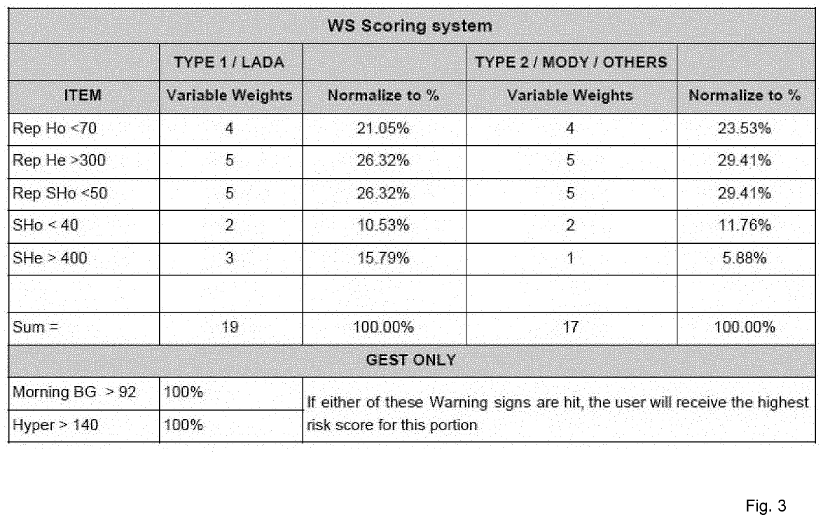

In another embodiment (e.g., for type 2 diabetes patients), Severe Hyperglycemic events (>400 mg/dl) may be weighted higher than Repeat Hypoglycemic events (<70 mg/dl). The weight values in FIG. 3 for Type 2 patients can, e.g., be changed as follows:

| Event | Weight |
|---|---|
| Rep Ho <70 | 1 |
| SHe >400 | 5 |

The WS may be applied as follows:
Within the set of readings, search for hypo events below 70 mg/dl, wherein two SMBG readings below 70 mg/dl in two consecutive days represent a hypo event. In order to determine if a hypo event occurs, repeated instances of the event are searched for. For example, the readings BG 67 (day 1), BG 56 (day 2), BG 50 (day 6), BG 65 (day 7) contain two events for BG below 70 mg/dl for 2 repeated days.
Within the set of readings, search for hyper events above 300 mg/dl, wherein a hyper event corresponds to more than three SMBG readings above 300 mg/dl.
Within the set of readings, search for Severe hypo events below 50 mg/dl, wherein two consecutive SMBG readings below 50 mg/dl in two days.
Within the set of readings, search for one very severe hypo event, corresponding to one reading below 40 mg/dl.
Within the set of readings, search for one very severe hyper event, corresponding to one reading above 400 mg/dl.

The WS weights are dependent on the diabetes type. For example, repeated high BG value in a diabetes type 1 patient might be a lot more or less important in comparison to the recommendation to a diabetes type 2 patient.

Thus, apart from dividing patients 5 into the groups G₁ and G₂, the patients 5 are also divided into the following diabetic types in order to determine the appropriate weights: type 1 diabetes, LADA (latent autoimmune diabetes in adults); type 2 diabetes, MODY (Maturity Onset Diabetes of the Young), NOTLISTED (diabetes not indicated), OTHERS; and GEST (gestational diabetes).

The weights (absolute and normalized as to sum up to 100%) for the group G₁ and each type are listed in FIG. 3. The table can also be used for identifying trends in the group G₂, but not for risk scoring. The weights were estimated and normalized for the scoring analysis as laid out above. Gestational diabetics (GEST) and pregnant diabetics constitute a distinct group. Gestational metrics are more stringent than all the other WS (as a mother and a baby are concerned). Therefore, their scoring is different from the other types (cf. the last two rows of the table in FIG. 3).

Referring to FIG. 3, gestational diabetics are scored based on the following metrics: low SMBG values (hypoglycemic events); high SMBG values (hyperglycemic events); morning fasting SMBG values above 92 mg/dl; and any hyperglycemic event in the day above 140 mg/dl. This scoring is similar to the groups G₁ and G₂ except for the last two items in FIG. 3. The last two metrics in the table in FIG. 3 are set up as in the WS case, except if the patient meets the requirements, they are assigned the highest possible risk score.

Each of the group G₁ and G₂ comprises metrics comparable to rank risk (high, low, stability, and daily average). The levels of risk are assigned as follows to the respective scores (score—risk): 0—No risk; 5—low risk; 2—medium risk; and 3—high risk.

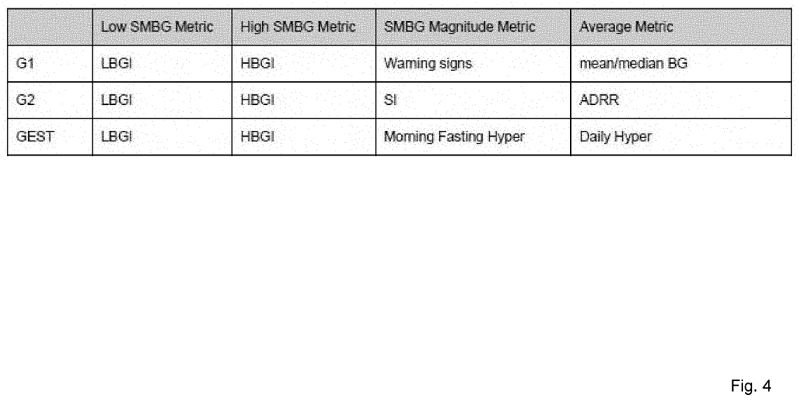

FIG. 4 shows a table summarizing the metrics applied for different groups G₁, G₂, and GEST of diabetic patients 5 for determining an individual risk score for the different groups and following the relative risk for lack of glycemic control. The term Morning Fasting Hyper refers to any BG value read before 9 AM and above 92 mg/dl. This generally corresponds to not eating (fasting) in the middle of the night. The term Daily Hyper refers to any BG value in the day above 140 mg/dl. These values are set very conservatively since they apply to a baby and a mother.

FIG. 5 shows a table summarizing the scoring system and comprising different risk metrics. For the different metrics such as LBGI, HBGI, and ADRR score criteria are depicted. Scores from 0 to 3 are applied, such scores referring to no risk (0), low risk (1), medium risk (2), and high risk (3).

In FIG. 5, A1C refers to (HbA1c). If a patient has the corresponding percentage of WS, they are assigned to the respective scoring category from 0 to 3. The median values for risk relate to hemoglobin A1C (HbA1C) below 7.0%, 8.0%, 9.0%, and greater than 9.0%.

An alternative scoring system is described in Table 1 above.

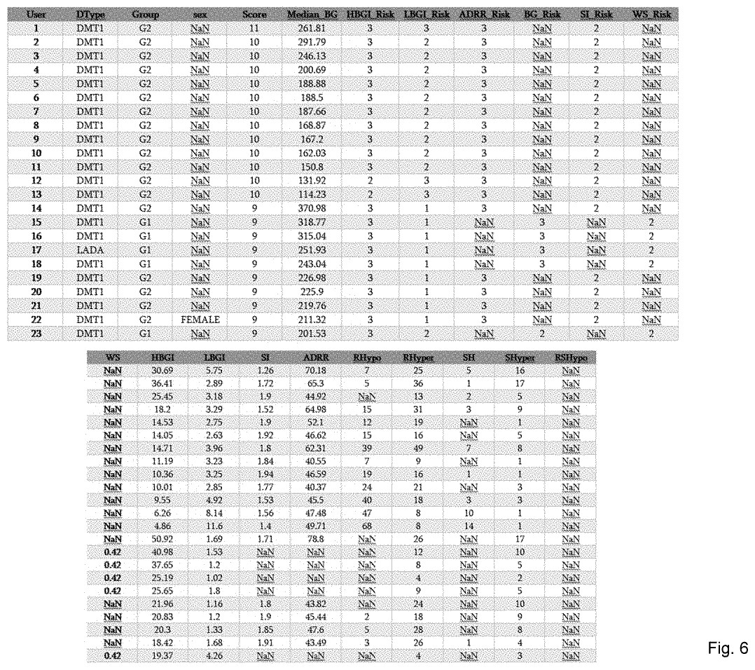

FIG. 6 shows a table comprising risk scores for 23 exemplary patients. The table is split in to an upper and a lower part for better display. The columns of the lower part continue the columns of the upper part of the table. The first column (“User”) indicates the 23 patients from 1 to 23. The risk scores for all four risk categories (see FIG. 5) were summed up for determining a total risk score (“Score” in FIG. 6—upper part in FIG. 6) for the groups G₁ and G₂. For example, the total risk score for patient 1 in FIG. 6 is 11 (score of 3 for “HGBI_Risk” plus score of 3 for BG Risk” plus score of 3 “ADRR Risk” plus score of 2 for “SI Risk”).

Different metrics have been applied for the two groups G₁ and G₂. For example, the metrics “ADRR Risk” (ADRR) and “SI Risk” (SI) have not been applied to the group G₁. For the group G₂, “BG Risk” and “WS Risk” (WS) have not been applied. Not applying or not taking into account some metric is identified by “NaN” in FIG. 6.

In the lower part of FIG. 6, actual values (not risk scores) are depicted for HBGI, LBGI, SI, WS, and ADRR. “RSHypo” corresponds to a repeated Severe Hypoglycemic event with BG<50 mg/dl, “RHypo” corresponds to a repeated Hypoglycemic event with BG<70 mg/dl, and “SH” corresponds to a Severe Hypoglycemic event with BG<40 mg/dl.

Subsequently, the patients 5 in the groups G₁ and G₂ were sorted from highest to lowest total score. The individual total risk determined for the groups G₁ and G₂ can provide health care providers can filter by “diabetic type” and “Risk Score” to see which patients 5 are in most need of support.

Warning signs WS of past days can be used for automated messaging, e.g., to a healthcare provider. For example, an automated message is sent each time a user experiences a severe hypo below 40 mg/dl. If over the course of the week (or specified timeframe), a patient reaches a certain threshold determined by the healthcare provider, the healthcare provider can be notified and contact the patient directly. This means that automated messaging or notification can help patients 5 with little need for support, whereas patients 5 with more substantial risk can be treated by the healthcare provider in person.

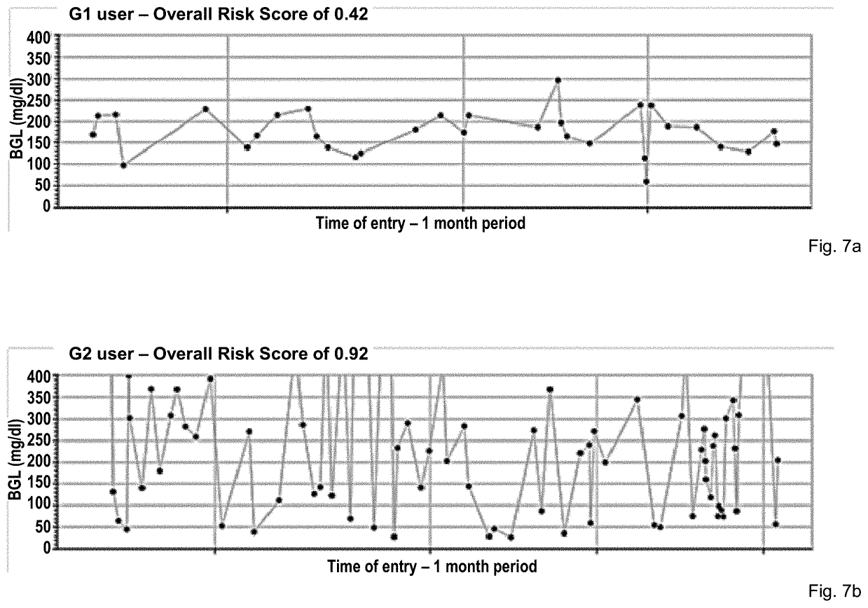

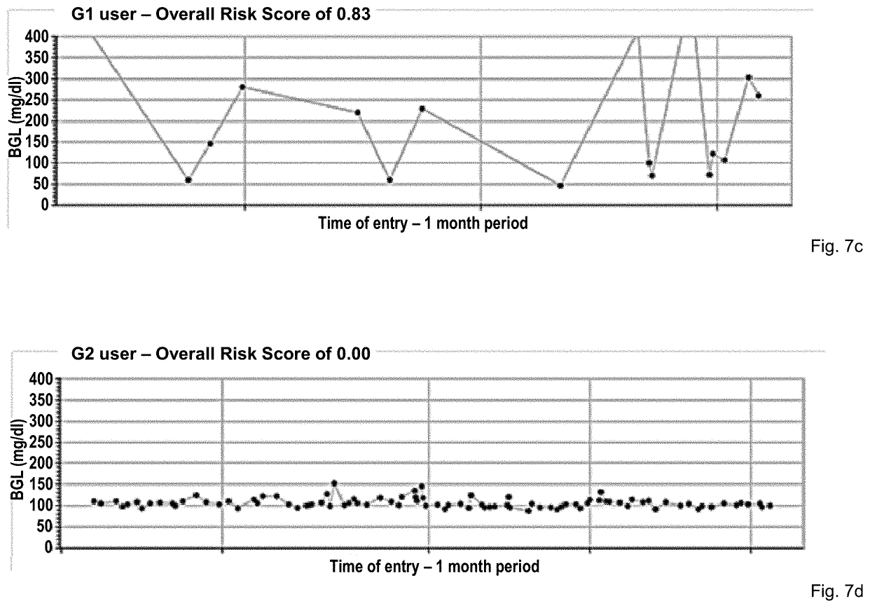

In FIGS. 7a to 7d, four graphs of BG values (SMBG reading) depending on time of recording or measuring for different patients are shown. Each graph represents one of four patients 5 in the following four groups and with the following total risk determined as described above: G₁—medium risk (FIG. 7a); G₂—high risk (FIG. 7b); G₁—high risk (FIG. 7c); and G₂—no risk (FIG. 7d). The desired range for BG values is between 70 mg/dl and 180 mg/dl.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A computer-implemented method for determining a relative risk for lack of glycemic control for at least two patients, comprising:
providing a first set of glucose readings assigned to a first patient, the first set of glucose readings collected by conducting a first frequency of measurements;
providing a second set of glucose readings assigned to a second patient, the second set of glucose readings collected by conducting a second frequency of measurements different from the first frequency of measurements;
providing a plurality of evaluation parameters in a data storage device, each of the evaluation parameters assigned to at least one of the first and second frequencies of measurements;
determining, depending on the first frequency of measurements, a first evaluation parameter from the plurality of evaluation parameters;
determining, depending on the second frequency of measurements, a second evaluation parameter from the plurality of evaluation parameters, the second evaluation parameter being different than the first evaluation parameter;
determining, from the first set of glucose readings, a first evaluation value assigned to the first patient for the first evaluation parameter;
determining, from the second set of glucose readings, a second evaluation value assigned to the second patient for the second evaluation parameter;
determining a first risk score assigned to the first patient and a second risk score assigned to the second patient from the first evaluation value and the second evaluation value, respectively; and
determining a relative risk for lack of glycemic control by comparing a first total risk score comprising the first risk score and a second total risk score comprising the second risk score, the relative risk indicating the risk for lack of glycemic control being higher for the first patient or the second patient.

2. The method according to claim 1, further comprising:
providing a first set of glucose readings collected in a first plurality of spot monitoring glucose measurements applying the first frequency of measurements; and/or
providing a second set of glucose readings collected in a second plurality of spot monitoring glucose measurements applying the second frequency of measurements.

3. The method according to claim 1, comprising, for at least one of the first and second sets of glucose readings:
determining one or more consecutive glucose measurement values collected after collecting a preceding glucose measurement value in a preceding measurement, the one or more measurement values collected in one or more consecutive measurements within a predetermined time window after the preceding measurement event; and
excluding the one or more consecutive glucose measurement values from the determining of the first and/or second evaluation value.

4. The method according to claim 1, further comprising:
determining, from the first set of glucose readings a plurality of first evaluation values assigned to the first patient for a plurality of first evaluation parameters; and
determining, from the second set of glucose readings, a plurality of second evaluation values assigned to the second patient for a plurality of second evaluation parameters;
wherein the first total risk score and the second total risk score are determined by summing first and second risk scores for the plurality of first evaluation values and the plurality of second evaluation values, respectively.

5. The method according to claim 4, wherein at least one common evaluation parameter is provided in both the plurality of first and the plurality of second evaluation parameters.

6. The method according to claim 1, wherein the first set of glucose readings is assigned to a first patient having a first type of diabetes and the second set of glucose readings is assigned to a second patient having a second type of diabetes, the method further comprising selecting at least one of the first evaluation parameter and the second evaluation parameter depending on the first type of diabetes and the second type of diabetes, respectively.

7. The method according to claim 1, wherein the first and second patients are different.

8. The method according to claim 1, wherein the first evaluation parameter and the second evaluation parameter are selected from the following group: low blood glucose index, high blood glucose index, warning signs, mean blood glucose, median blood glucose, stability index, and total daily blood glucose variation determined by summing up peak risks of hypoglycemia and hyperglycemia events per day.

9. The method according to claim 8, wherein the first evaluation parameter or the plurality of first evaluation parameters is selected from the following group: low blood glucose index; high blood glucose index, warning signs, mean blood glucose, and median blood glucose.

10. The method according to claim 8, wherein the second evaluation parameter or the plurality of second evaluation parameters is selected from the following group: low blood glucose index; high blood glucose index, stability index, and total daily blood glucose variation determined by summing up peak risks of hypoglycemia and hyperglycemia events per day.

11. The method according to claim 4, wherein:
the plurality of first evaluation parameters comprises low blood glucose index (LBGI); high blood glucose index (HBGI), warning signs (WS), and median blood glucose (median),
the plurality of second evaluation parameters comprises low blood glucose index (LBGI); high blood glucose index (HBGI), stability index (SI), and total daily blood glucose variation determined by summing up peak risks of hypoglycemia and hyperglycemia events per day (ADRR),
the risk scores for the plurality of first values and the plurality of second values, respectively, are determined as follows:

| | 0 (NO | 1 (LOW | 2 (MEDIUM | 3 (HIGH |
|---|---|---|---|---|
| Risk scores | RISK) | RISK) | RISK) | RISK) |
| LBGI | ≤1 | >1 & ≤2.5 | >2.5 & ≤5 | >5 |
| HBGI | ≤1 | >1 & ≤4.5 | >4.5 & ≤9 | >9 |
| SI | ≥3 | <3 & ≥2 | <2 & ≥1 | <1 |
| ADRR | ≤19 | >19 & ≤30 | >30 & ≤40 | >40 |
| Warning signs | ≤15% | >15% & ≤40% | >40% & ≤65% | >65% |
| Median | ≥70 & ≤140 | >140 & ≤154 | >154 & ≤169 | >169 |
| | | | or <70 | |

the parameter value for warning signs is calculated as warning sign risk variable (WSRV)
$$W S R V = \frac{{\sum }_{i} ( {w}_{i} \cdot {x}_{i} )}{{\sum }_{i} {w}_{i}} * 100 ,$$
wherein index i denotes events, weight for the event with index i is denoted with wᵢ, and variable xᵢ represents a Boolean value whether (Boolean value=1) or not (Boolean value=0) the event has occurred.

12. The method according to claim 1, wherein at least one of the first total risk score and the second total risk score being above a predefined threshold value is indicative of a need for therapy review and/or therapy adjustment for the first patient and the second patient, respectively.

13. The method according to claim 1, further comprising at least one of the following steps:
a) predefining a first maximum achievable total risk score and a first threshold value above 0.5, wherein when the ratio of the first total risk score and the first maximum achievable total risk score is above the first predefined threshold, providing for at least one of
indicating at least one of need for therapy review and therapy adjustment for the first patient, and
triggering an electronic message to be sent to the first patient, and
b) predefining a second maximum achievable total risk score and a second threshold value above 0.5, wherein when the ratio of the second total risk score and the second maximum achievable total risk score is above the second predefined threshold, providing for at least one of
indicating at least one of need for therapy review and therapy adjustment for the first patient, and
triggering an electronic message to be sent to the first patient.

14. A system for determining a relative risk for lack of glycemic control for at least two patients, the system comprising a data processer configured to:
provide a first set of glucose readings assigned to a first patient, the first set of glucose readings collected by conducting a first frequency of measurements;
provide a second set of glucose readings assigned to a second patient, the second set of glucose readings collected by conducting a second frequency of measurements different from the first frequency of measurements;
provide a plurality of evaluation parameters in a data storage device, each of the evaluation parameters assigned to at least one of the first and second frequencies of measurements;
determine, depending on the first frequency of measurements, a first evaluation parameter from the plurality of evaluation parameters;
determine, depending on the second frequency of measurements, a second evaluation parameter from the plurality of evaluation parameters, the second evaluation parameter being different than the first evaluation parameter;
determine, from the first set of glucose readings, a first evaluation value assigned to the first patient for the first evaluation parameter;
determine, from the second set of glucose readings, a second evaluation value assigned to the second patient for the second evaluation parameter;
determine a first risk score assigned to the first patient and a second risk score assigned to the second patient from the first evaluation value and the second evaluation value, respectively; and
determine a relative risk for lack of glycemic control by comparing a first total risk score comprising the first risk score and a second total risk score comprising the second risk score, the relative risk indicating the risk for lack of glycemic control being higher for the first patient or the second patient.

15. A non-transitory computer readable medium having stored thereon computer-executable instructions for performing the method according to claim 1.
